(19) United States
(12) Patent Application Publication
(10) Pub. No.: US 2016/0097756 A1
(43) Pub. Date: Apr. 7, 2016

(54) SYSTEMS AND METHODS FOR MAPPING AN EXPLOSIVE EVENT

(71) Applicant: BlackBox Biometrics, Inc., Rochester, NY (US)

(72) Inventors: David A. Borkholder, Rochester, NY (US); Jeffrey L. Rogers, Rochester, NY (US)

(73) Assignee: BlackBox Biometrics, Inc., Rochester, NY (US)

(21) Appl. No.: 13/838,492

(22) Filed: Mar. 15, 2013

Related U.S. Application Data
(60) Provisional application No. 61/708,883, filed on Oct. 2, 2012.

(51) Int. Cl. G01N 33/22

(52) U.S. Cl. CPC G01N 33/227

(57) ABSTRACT

Systems and methods for mapping an explosive event are discussed. In an example, a system for mapping an explosive event can include a plurality of sensor devices and a data analysis system. Each of the plurality of sensor devices can include an environmental sensor to measure at least one parameter of an explosive event. Each sensor device can also include a time synchronization circuit to synchronize time stamping of the parameter measured by the environmental sensor, and a wireless communication interface. The data analysis system can access measurement data generated by the plurality of sensor devices and analyze the measurement data to produce a visualization mapping the measurements onto a virtual representation of the test environment.

### RELATED APPLICATIONS

This application claims the benefit of U.S. Provisional Application No. 61/708,883, filed Oct. 2, 2012, and titled “SYSTEMS AND METHODS FOR MAPPING AN EXPLOSIVE EVENT,” which application is incorporated herein by reference in its entirety.

### TECHNICAL FIELD

This application relates generally to collecting and processing (e.g., analyzing) environmental sensor data, and more specifically to systems and methods for dynamic mapping explosive event data for example using time synchronized high-fidelity sensors or a dense meshwork of time synchronized low-fidelity sensors.

### BACKGROUND

Understanding how an explosive blast affects structures, vehicles, and people is important to designing safer equipment for military and paramilitary uses. Traditionally, blast testing with vehicles or protective clothing involves use of expensive sensors and data collection equipment. The traditional equipment is difficult and time consuming to setup and only provides a limited amount of data. While the data tends to be very precise, the expense and difficulty involved in setting up the sensors and collection equipment usually limits the number of collection points and number of tests that can realistically be performed. The precision sensors are designed to accurately measure explosive blasts in a specific orientation, but accuracy deteriorates in complex environments and situations with reflected waves. Accurate capture of complex blast waveforms is difficult to achieve with traditional sensors.

### BRIEF DESCRIPTION OF THE DRAWINGS

Some embodiments are illustrated by way of example and not limitation in the figures of the accompanying drawings in which:

FIG. 1 is a block diagram depicting a sensor device, according to an example embodiment, that can be used for mapping an explosive event.

FIGS. 2A-2C are illustrations of a sensor device, according to an example embodiment, that can be used for mapping an explosive event.

FIGS. 3A-3B are block diagrams illustrating two sensor configurations, according to example embodiments, that can be used in mapping explosive events.

FIG. 4 is an illustration depicting illustrating a plurality of sensors, according to an example embodiment, within an area to be mapped during an explosive event.

FIG. 5 is an illustration depicting illustrating multiple sensor grids, according to an example embodiment, laid out within an area to be mapped during an explosive event.

FIG. 6 is a flowchart illustrating a method, according to an example embodiment, for mapping an explosive event.

FIG. 7 is a flowchart illustrating a method, according to an example embodiment, for mapping an explosive event.

FIG. 8 is a flowchart illustrating a method, according to an example embodiment, for dynamically mapping an explosive event using data from a plurality of sensor devices.

FIG. 9 is a diagrammatic representation of a machine in the example form of a computer system within which a set of instructions for causing the machine to perform any one or more of the methodologies discussed herein may be executed.

### DEFINITIONS

Real-time—For the purposes of this specification and the associated claims, the term “real-time” is used to refer to calculations or operations performed on-the-fly as events occur or as input is received by the operable system. However, the use of the term “real-time” is not intended to preclude operations that cause some latency between input and response, so long as the latency is an unintended consequence induced by performance characteristics of the machines (e.g., computers) involved in the operation.

### DETAILED DESCRIPTION

Example systems, apparatus, and methods for mapping explosive events are described. The systems and methods for mapping explosive events in some example embodiments may provide numerical and visual analysis of a blast event within a target environment. In some examples, the target environments can include buildings or other structures, vehicles, and people (usually represented by instrumented test subjects). In an example, a target test environment can be instrumented with a large number of discrete sensor devices, which can be time synchronized to enable dynamic mapping of an explosive event. In the following description, for purposes of explanation, numerous specific details are set forth in order to provide a thorough understanding of example embodiments. It will be evident, however, to one skilled in the art that the present invention may be practiced without these specific details. It will also be evident that mapping of explosive events is not limited to the examples provided and may include other scenarios not specifically discussed.

In accordance with an example embodiment, a plurality of environmental sensors can be located throughout a target area (environment). The sensors can be completely self-contained devices capable of recording blast or pressure waves, among other things. After an explosive event is triggered within the target environment, the sensors can each be retrieved and interrogated to obtain raw blast data for further analysis. In certain examples, the sensors can wirelessly transmit results after a blast event has been detected. Locations associated with each sensor device can be mapped manually or sufficiently precise location data can be obtained automatically by each sensor device. Automatic location data can be obtained via triangulation, GPS, or any other suitable means. In some examples, locations of the environmental sensors can be determined via triangulation with external signals or via signals generated by the individual sensors. In an example, images of the test environment can be taken and subsequently processed via various forms of image processing to determine sensor locations. In this example, the sensor devices can be coated with infra-red reflective or other suitable material to allow for easy identification within captured images.

Each of the sensor devices can capture data such as pressure, velocity, air density, and acceleration (linear or rotational). In an example, the sensor devices can be stand-alone battery operated devices that can be placed and operated by non-technical personal. In certain examples, the sensor devices operate within the target environment completely wirelessly, but may be connected via wires for power and data collection purposes. In another example, the sensors can be wired into a grid of sensors with no or limited wireless communication capabilities. In yet other examples, the sensors can include passive wireless communication capabilities, such as near-field-communication (NFC).

In certain examples, the sensor devices can individually provide high-fidelity environmental data, such as pressure, temperature, or accelerations, which can subsequently be transformed into dynamic maps illustrating time-based propagation of blast characteristics within the test environment. In other examples, a dense meshwork of low-fidelity sensors can be utilized to capture blast data throughout the test environment. A meshwork of sensors can result in a single sheet or blanket of interconnected sensors that can be placed within a test environment. Locations of the low-fidelity sensor can be determined based on place of the meshwork the sensors are attached to, or by means such as triangulation with external signals or via signals generated by the individual sensors. In an example, localized averaging of sensor data from the low-fidelity sensors can be used to obtain a courser grid of high-fidelity data from the meshwork of low-fidelity sensors.

The use of multiple sensor devices within a target environment can allow explosive blast mapping including blast propagation and event levels at specific locations throughout the test environment, among other things. For example, explosive blast mapping can be used to determine where breachers should shelter in a given environment. Further, explosive blast mapping can be used to determine which breacher is least likely to suffer cognitive impairment, thus should be first to enter a room. In an example, sensor devices can be placed in 1 meter increments on walls, floors, and ceilings as well as around corners, behind posts, and at different elevations. In an example using a dense meshwork of low-fidelity sensors, sensors can be positioned as frequently as every centimeter throughout the test environment. When test subjects are used, sensors can also be placed on them to measure exposure.

The sensors and explosive blast mapping in an environment can yield insights into exposures leading to a plurality of injuries (e.g., traumatic brain injury, ocular injuries, or hearing related injuries) and other blast related medical conditions. Sensing the environment in explosive blast can lead to better design of vehicles or structures and can lead to an understanding of how explosive blast exposure impacts the health of nearby people.

Each sensor device can provide data precisely time stamped based on a high precision internal clock to permit calculation of propagation velocities and overlay of captured sensor data for mapping. In certain examples, the sensor devices can use wireless clock synchronization from a base station or by using one of the sensor devices as a sync source.

Sensor device uniformity can be one factor in delivering useful explosive blast mapping. Co-pending application U.S. patent application Ser. No. 13/371,202, titled “Event Monitoring Dosimetry Apparatuses and Methods Thereof,” which is hereby incorporated by reference in its entirety, addresses some methods of achieving low-cost uniform sensor devices.

### Example Sensor Devices

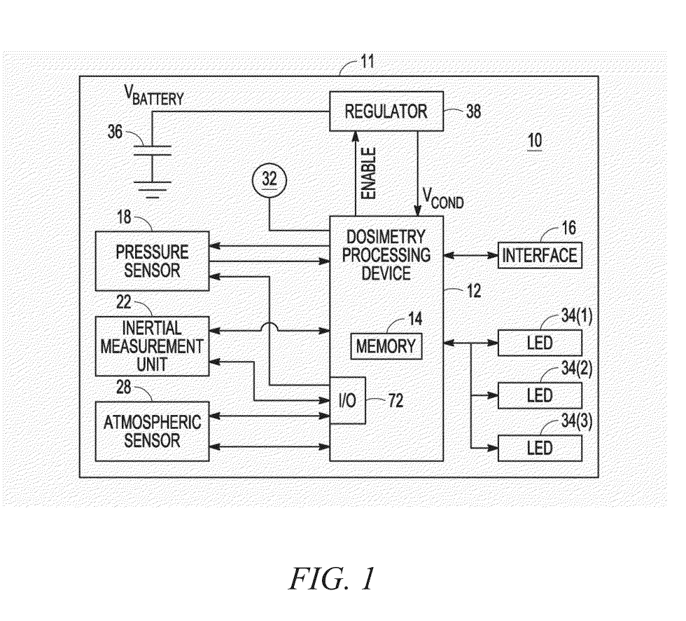

FIG. 1 is a block diagram depicting a sensor device 10 (also referred to as monitoring dosimetry apparatus 10) that can be used for mapping an explosive event, according to an example embodiment. An exemplary event monitoring dosimetry apparatus 10 is illustrated in FIG. 1. The event monitoring dosimetry apparatus 10 includes a housing assembly 11 with a dosimetry processing device 12 with a memory 14, an interface device 16, a pressure sensor 18, a cover 20(1), an inertial monitoring unit 22, shock mounting system 24, mounting system 26, an atmospheric sensor 28, a power system 30, an engagement device 32, and a series of different colored LEDs with different numeric indicators 34(1)-34(3), although the apparatus 10 could include other types and numbers of systems, devices, components and elements in other configurations. This technology provides a number of advantages including providing a more effective and efficient event monitoring dosimetry apparatus. In certain examples, the apparatus 10 can also include wireless communication circuitry, such as WiFi (e.g., IEEE 801.11 type networking) or near-field communication (NFC) circuitry. In certain examples, the NFC circuitry can be passive, limiting the amount of power required within the device to power communications.

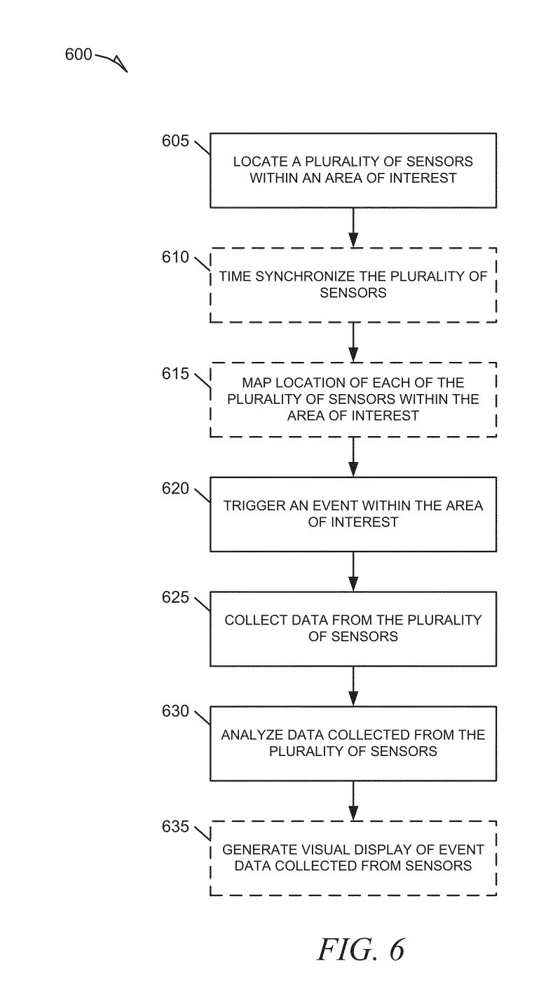

Referring more specifically to FIG. 1, the dosimetry processing device 12 can comprise one or more processors internally coupled to the memory 14 by a bus or other links, although other numbers and types of systems, devices, components, and elements in other configurations and locations can be used. The one or more processors in the dosimetry processing device 12 executes a program of stored instructions for one or more aspects of the present technology as described and illustrated by way of the examples herein, although other types and numbers of processing devices and logic could be used and the processor could execute other numbers and types of programmed instructions. The memory 14 in the dosimetry processing device 12 stores these programmed instructions for one or more aspects of the present technology as described and illustrated herein, although some or all of the programmed instructions could be stored and executed elsewhere. A variety of different types of memory storage devices, such as a solid state memory, can be used for the memory 14 in the dosimetry processing device 12. The flow chart shown in FIG. 6 is representative of example steps or actions of this technology that may be embodied or expressed as one or more non-transitory computer or machine readable instructions stored in memory 14 that may be executed by the one or more processors.

The interface device 16 in the dosimetry processing device 12 is used to operatively couple and communicate between the dosimetry processing device 12 and one or more external computing or storage devices, although other types and numbers of communication networks or systems with other types and numbers of connections and configurations can be used.

Although an example of the dosimetry processing device 12 is described herein, it can be implemented on any suitable computer system or computing device. It is to be understood that the devices and systems of the examples described herein are for exemplary purposes, as many variations of the specific hardware and software used to implement the examples are possible, as will be appreciated by those skilled in the relevant art(s).

The pressure sensor 18 is coupled to the dosimetry processing device 12, although the pressure sensor 18 could be coupled to other types and numbers of devices. In this example, the pressure sensor 18 is a single pressure sensor to help achieve low cost and disposability parameters, although other types and numbers of pressure sensors could be used. Additionally, in this example the pressure sensor 18 is insensitive to direction and will substantially detect the same pressure waveform independent of the relative orientation of the pressure sensor 18 to the pressure front, although the pressure sensor could have other degrees of improved directional sensitivity, such as fifty percent or more by way of example only. To have multi-directional sensitivity, in this example the pressure sensor 18 is positioned below a wire mesh dome cover 20(1) which extends out and away from the housing 11, although other types of covers could be used to enable multi-directional sensing by the pressure sensor 18.

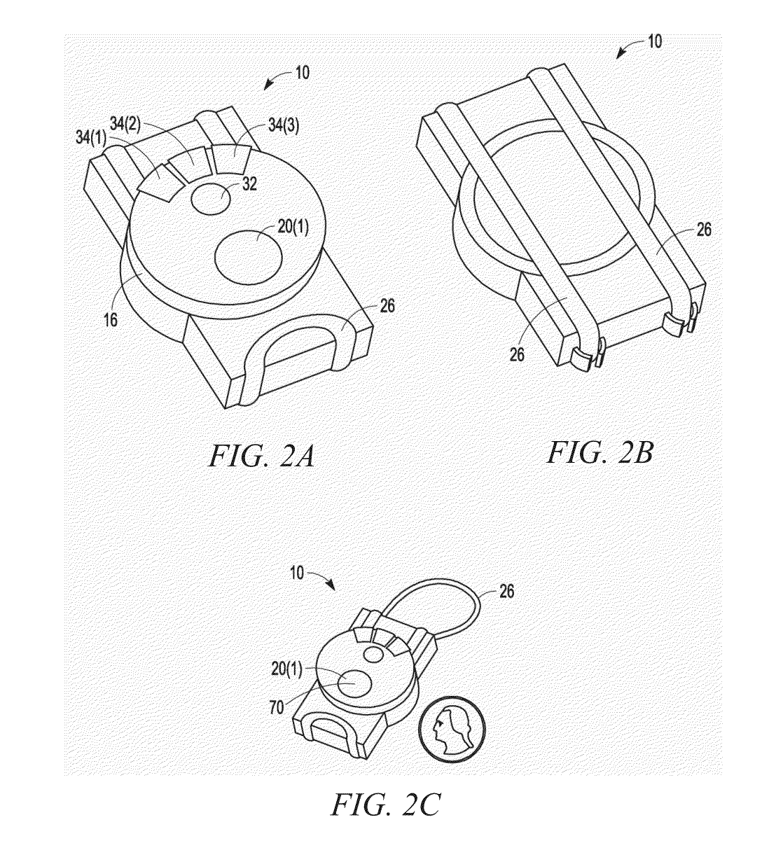

The mesh dome cover 20(1) comprises a very fine mesh layer, which acts as a filter for particles, sandwiched between two larger mesh layers used for structural strength, although the mesh dome cover 20(1) can be made with other types and numbers of layers. Additionally, the mesh dome cover 20(1) can be made of a variety of different types of materials, such as metallic materials, non-metallic materials, and fabric weaves as well as combinations of different materials by way of example only. Further, a floating body, such as a sphere or beads which are loose, other structures such as beads which are packed in and cannot move or a porous matrix structure also may also be inserted within the mesh dome cover 20(1) to improve omni-directionality. Further, a check valve 70 can be situated within the mesh dome cover 20(1) to improve omni-directionality as shown in FIG. 2C.

Referring back to FIG. 1, the inertial measurement unit 22 is a low-g (for example 16 g) three-axis accelerometer to capture linear acceleration in three axes, although other types (such as a high-g accelerometer, for example >100 g) and numbers of inertial measurement units could be used. In an example, the inertial measurement unit 22 can include two (or more) three axis accelerometers. In another example, the inertial measurement unit 22 could be a gyroscope which records rotational acceleration. To account for differences in pressure readings from the pressure sensor 18 which depends on the incident direction of the force, the three-axis acceleration information from the inertial monitoring unit 22 can be used by the dosimetry processing device 12 to determine the vector of movement coincident with the arrival of the pressure shock front. This indicates the relative angle of the dosimetry apparatus 10 to the force allowing for compensation of the measured pressure profile including the levels of the stored reading thresholds to improve accuracy and precision with respect to the obtained readings and the identification of events.

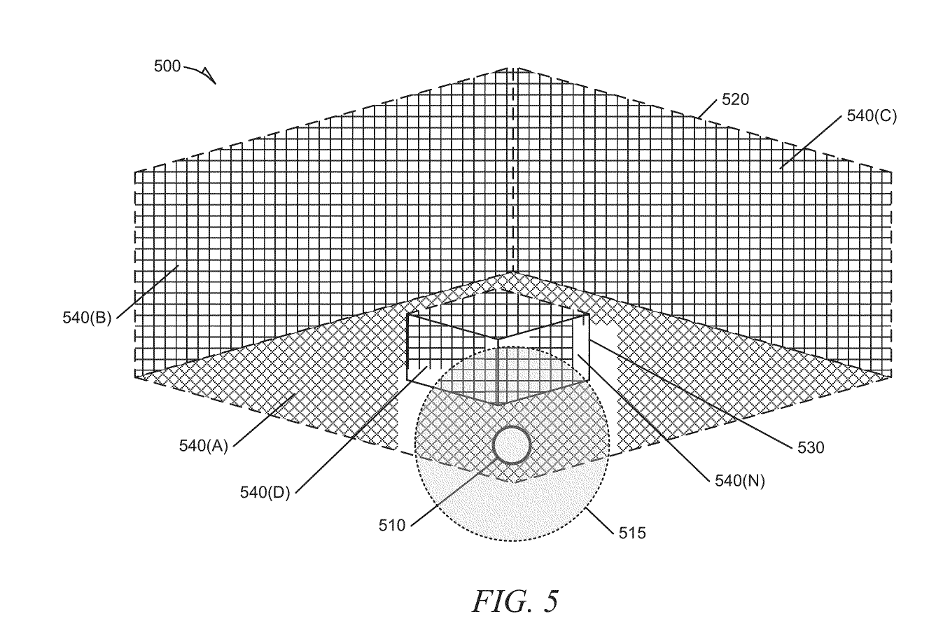

Referring to FIG. 2C, the mounting system 26 is used to mount the housing 11 of the dosimetry apparatus 10 to an entity, such as an object or person. In this example, the mounting system 26 comprises a strap that can be mounted to a helmet strap or fabric webs on uniforms, although other types of mounting systems 26 could be used. The mounting system 26 can be adapted for use in mounting the dosimetry apparatus 10 into a mesh of dosimetry apparatuses, such as illustrated in FIG. 5.

By way of example only, other types of mounting systems 26 may include adhesive, hook and loop mating fasteners, fabric wraps with hook and loop mating fasteners, elastic straps with or without snaps, hooks, grommet snaps embedded in the housing and attached to a helmet strap, and non-elastic straps with a tightening mechanism. The manners by which these attachment mechanisms operate are well known to those of ordinary skill in the art and thus will not be described in detail here. For example, the elastic strap can comprise an elastic loop that hooks onto a portion of the housing 11, wraps around the desired entity, and attaches to another portion of the housing to secure the housing 11.

The atmospheric sensor 28 is coupled to the dosimetry processing device 12 and provides atmospheric readings within the dosimetry apparatus 10, although other types and numbers of atmospheric monitors could be used and the atmospheric sensor 28 could be positioned to take other readings.

The power system 30 includes a battery 36 coupled to a regulator 38 which is coupled to the dosimetry processing device 12, although other types of power systems with other types and numbers of components, such as one with an energy harvester and/or without a regulator 38 could be used. The regulator 38 is coupled to regulate power provided by the battery 36 to the dosimetry processing device 12. Additionally, in this example power for the pressure sensor 18, the inertial measurement unit 22, the atmospheric sensor 28, and/or the strain gauge 29 is coupled directly from the dosimetry processing device 12 to save power, although other types and numbers of devices and systems could be coupled directly to the dosimetry processing device 12 to provide power. Additional, power management may be achieved through stored programmed instructions executed by the dosimetry processing device 12 for a standard monitoring mode, a lower power monitoring mode and a sleep mode, although other types and numbers of modes can be used. The inertial measurement unit 22 provides data to the dosimetry processing device 12 to identify periods of inactivity to trigger the lower power standby mode when minimal activity is identified or sleep mode when prolong periods of inactivity are identified for power savings. Activity sensing by the inertial measurement unit 22 enables the dosimetry processing device 12 to switch to standard monitoring mode with a high sampling rate for the inertial measurement unit 22 and pressure sensor 18 by way of example. In this example, all peripheral devices, such as pressure sensor 18, inertial measurement unit 22, and atmospheric sensor 28 by way of example only, are powered through digital input/output (I/O) pins 72 on the processor in the dosimetry processing device 12 rather than from a power bus. This allows peripheral devices to be turned off via the processor in the dosimetry processing device 12 saving power. The regulator 38 is enabled through an I/O input/output (I/O) pin on the processor in the dosimetry processing device 12. This allows the dosimetry processing device 12 to be completely turned off to extend shelf life. The dosimetry processing device 12 is activated either through the button or powering through USB.

The engagement device 32, such as a button by way of example only, is coupled to the dosimetry processing device 12, although the engagement device could be coupled in other manners. The engagement device 32 can be used to request an output of readings including of identified events, stored events and/or assessments of the readings. Additionally, other types and numbers of mechanisms for engaging the dosimetry processing device 12 can be used, such as another computing device coupling to the dosimetry processing device 12 through the interface 16 to request and obtain output data and other information, download a time and date stamp, set and/or reprogram criteria and other parameters by way of example only. In certain examples, an NFC chip can be incorporated for engaging the dosimetry processing device 12.

The series of different colored LEDs with different numeric indicators 34(1)-34(3) are used to provide a status indication for the output stored readings and of the assessment of the stored readings associated with identified events to provide immediate triage of the severity of an event, although other types and numbers of displays which provide other types of outputs can be used. In this example, LED 34(1) is green colored and has a numeric indicator of zero, LED 34(2) is yellow colored and has a numeric indicator of one, and LED 34(3) is red colored and has a numeric indicator of two, although other colors and symbols could be used.

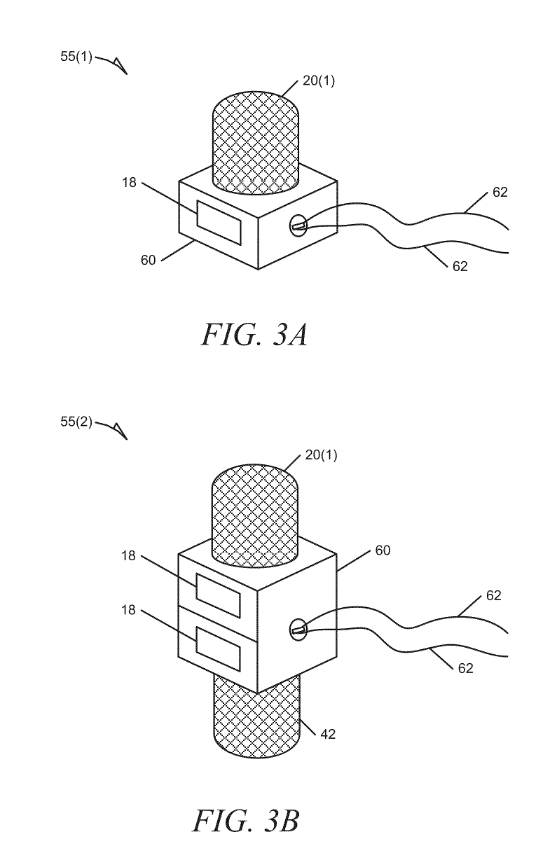

Referring to FIGS. 3A-3B examples of dosimetry pressure sensing apparatuses 55(1) and 55(2) are illustrated. The dosimetry pressure sensing apparatuses 55(1) and 55(2) are similar to the dosimetry apparatus 10, except as described and illustrated herein. In this example, the dosimetry pressure sensing apparatus 55(1) has a pressure sensor 18 in a housing 60 with a cover 20(1) seated over the pressure sensor 18, although the dosimetry pressure sensing apparatuses could have other numbers and types of system, devices and elements in other configurations. Additionally, the dosimetry pressure sensing apparatuses 55(1) has conductors 62 coupled to the pressure sensor 18 extending out from the housing 60 to couple to an external processing device as opposed to the internal processing device 12 in dosimetry apparatus 10. Additionally, in this example, the dosimetry pressure sensing apparatus 55(2) has two pressure sensors 18 on opposing sides of the housing 60 with a cover 20(1) seated over each of the pressure sensors 18, although the dosimetry pressure sensing apparatuses could have other numbers and types of system, devices and elements in other configurations, such as other locations for and numbers of pressure sensors. Additionally, the dosimetry pressure sensing apparatuses 55(2) has conductors 62 coupled to each of the pressure sensors 18 extending out from the housing 60 to couple to an external processing device as opposed to the internal processing device 12 in dosimetry apparatus 10.

### Example Operating Environment

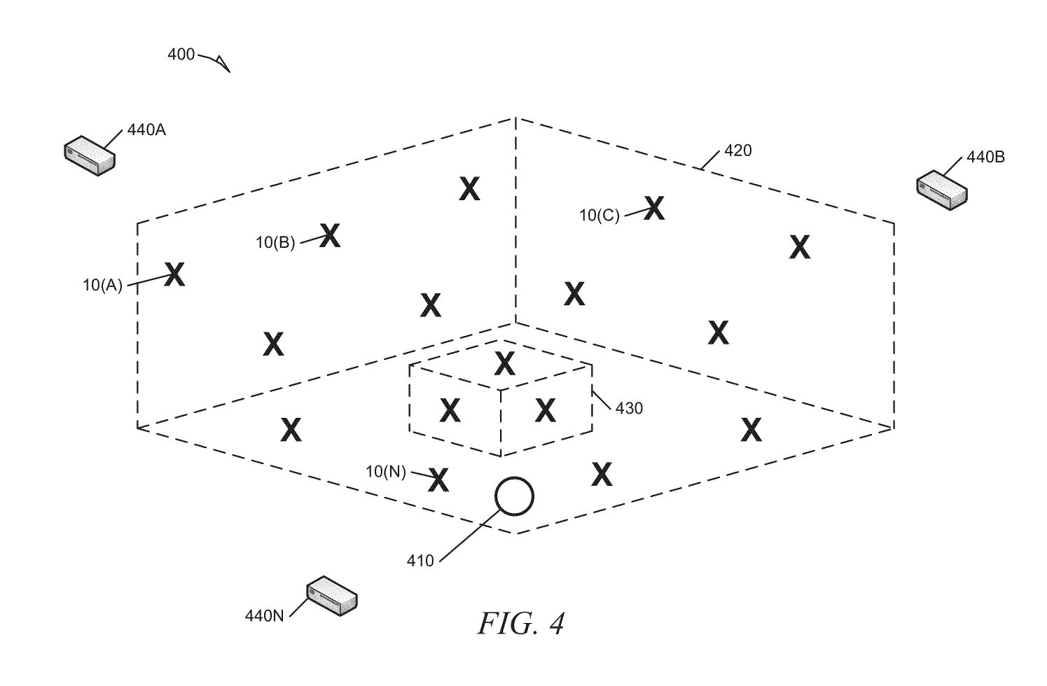

FIG. 4 is an illustration depicting a plurality of sensors (10(A)-10(N)) within an area 420 to be mapped during an explosive event, according to an example embodiment. FIG. 4 illustrates an example system 400 for mapping a target area, such as area 420. The system 400 can include a plurality of sensor devices 10(A) through 10(N) (collectively referred to as sensor device 10) depicted within the FIG. 4 by bold Xs. For the sake of clarity, not all of the bold Xs are labeled, but each of the illustrated Xs can represent an individual sensor device. Example systems can use more or fewer individual sensor devices depending on the resolution of measurements desired for a particular test. The target area 420 can include obstacles or other structures, such as structure 430. In certain embodiments, structure 430 can be a vehicle, blast barrier or similar structure undergoing explosive blast testing. The target area 420 illustrated in FIG. 4 is a simple room for clarity of illustration. The target area 420 can include more complex environments, such as hallways, corners, and doorways, among others. In fact, mapping explosive tests using the methods and systems discussed herein within more complex environments may illustrate some of the advantages of using a large number of discrete sensor devices to more accurately capture the effects of pressure waves reflecting off surfaces, among other things.

In certain examples, the system depicted in FIG. 4 can include one or more wireless base stations (440A through 440N, collectively referred to as wireless base station 440). The wireless base stations (440A-440N) can provide for communications, time synchronization, as well as location tracking via triangulation. The system 400 can use three or more wireless base stations (440A-440N) to triangulate individual positions of each of the sensor devices placed within the target environment 420. Triangulation can be performed on each sensor device, or alternatively through an external system (not depicted) collecting data obtained by each of the wireless base stations from each of the sensor device, such as through each device outputting one or more location beacon signals uniquely identifying the device.

Within system 400, device 410 is representative of a blast device to be triggered within the target area 420. Once the device 410 is triggered, the sensor devices 10 will record data associated with the explosive blast to be collected and analyzed later. Data collection can occur wirelessly via the wireless base stations, through wired connections to each sensor device, or via short range wireless protocols, such as NFC.

FIG. 5 is an illustration depicting multiple sensor grids (540(A)-540(N)) laid out within an area 520 to be mapped during an explosive event by system 500, according to an example embodiment. The system 500 includes a target area 520 covered by sensor grids or fabrics 540A-540N, collectively referred to as sensor grid (or grids) 540. In an example, a sensor grid or fabric is composed of a large number of relatively closely spaced sensor devices, such as sensor device 10. The idea behind the sensor grids is to enable the use of statistical analysis to account for destroyed sensors and/or low accuracy or resolution sensors, while also providing for a greater degree of data coverage across an area. In an example, the sensor grid 540 can contain sensors spaced at 1 cm to 1 m intervals. The grid array size is dependent on spacing and area coverage, but can consist of tens to thousands of sensor devices.

In this example, the explosive device 510, when triggered, may destroy a certain percentage of sensors within the area 515. However, due to the density of the sensor grids 540, statistical analysis can extrapolate data from neighboring sensors to provide meaningful data across the covered areas.

### Example Methods

FIG. 6 is a flowchart illustrating a method 600 for mapping an explosive event, according to an example embodiment. In an example, the method 600 can include operations such as: locating a plurality of sensors at 605, optionally time synchronizing the plurality of sensors at 610, optionally mapping sensor locations at 615, triggering an event at 620, collecting data from the sensors at 625, analyzing data collected from the sensors as 630, and optionally generating visual displays of the event data at 635.

In this example, the method 600 can begin at 605 with a plurality of sensors, such as sensor device 10, being distributed throughout the target environment. At 610, the method 600 can optionally include a time synchronization to ensure all sensors are collecting data synchronized to a common time. Time synchronization can be important to ensure data analysis is comparing and contrasting data points that occurred at the same point in time. Time synchronization across the plurality of sensors positioned throughout a test environment can allow for visualization of blast propagation throughout the environment.

Different methods of time synchronization can be used within a network of sensors, such as illustrated in FIG. 4 and FIG. 5. If the sensor devices include GPS circuitry, a GPS signal can be used to synchronize the sensors. Alternatively, one of the sensor devices can initiate a synchronization signal that can be broadcast or propagated throughout the entire network of sensor devices. In an example, a Flooding Time Synchronization Protocol (FTSP) can be used for time synchronization. FTSP utilizes a single broadcasted message to obtain time synchronization reference points between the sender and its neighbors. Nodes broadcast time synchronization messages periodically and synchronize their clocks to that of an elected leader. The algorithm is robust; it handles topology changes and node failures well.

At 615, the method 600 can optionally continue with the location of each of the plurality of sensors being determined via triangulation, image analysis, or other similar means. In certain examples, the locations of the plurality of sensor devices can be manually entered into a processing system at 615. In examples using triangulation, triangulation can be performed using external base station devices, or can be done using signals generated by individual sensor devices.

At 620, the method 600 can continue with an explosive device, such as device 410, being triggered within the target environment. At 625, the method 600 can continue with a processing system collecting data from the plurality of sensors surviving the explosive event. Data collection can be via a wired or wireless connection between a data analysis system and the sensor devices.

At 630, the method 600 can continue with the processing system analyzing data collected from the plurality of sensors exposed to the event triggered in operation 620. The data analysis performed in operation 630 can include mapping event data collected from the plurality of sensors in a virtual 3-D environment to visualize the captured result of the event. At 635, the method 600 can optionally conclude with the processing system generating visual display of the collected event data. As discussed previously, the event data and resulting visualizations can include pressure data, velocity data, air density data, and acceleration data captured by the individual sensors. The data visualizations can also include visualization depicting how one or more parameters changed over time. For example, data visualizations can include a 2D or 3D false color map of overpressure evolution over time, with data interpolation used to estimate values between measurement points. This visualization can utilize sensor device position data, manually entered or dynamically generated by the system, and time synchronization between sensor devices. The visualization can highlight regions above a threshold for a given measurement (e.g. pressure, acceleration) or calculated (e.g. impulse, velocity) to identify regions of interest (e.g. safety, equipment damage, etc). The data interpolation can operate on individual sensor device data or data averaged across multiple lower-resolution sensor devices in a mesh configuration (such as illustrated in FIG. 5) to produce high-resolution data for mapping and visualization. Data interpolation can be used to fill-in gaps in areas where sensor devices were destroyed or failed to function properly. In certain examples, data interpolation can be performed between sensor devices that are spatially distant (e.g., across a room or hallway). In these examples, interpolation between spatially distant sensor devices can be used for 3D mapping of the test environment.

Though arranged serially in the example of FIG. 6, other examples may reorder the operations, omit one or more operations, and/or execute two or more operations in parallel using multiple processors or a single processor organized as two or more virtual machines or sub-processors. Moreover, still other examples can implement at least some of the operations as one or more specific interconnected hardware or integrated circuit modules with related control and data signals communicated between and through the modules. Thus, any process flow is applicable to software, firmware, hardware, and hybrid implementations.

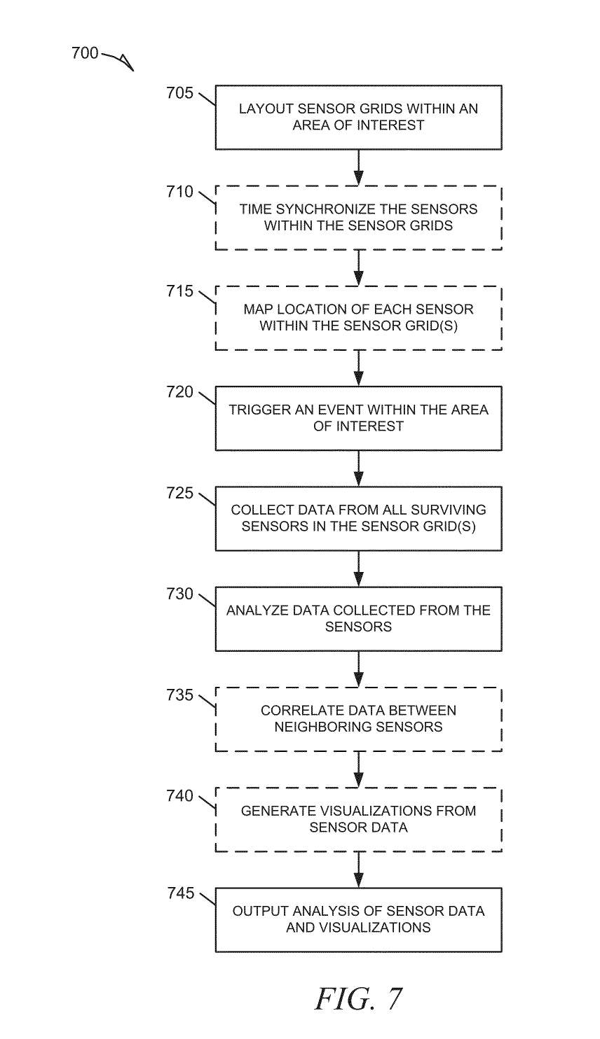

FIG. 7 is a flowchart illustrating a method 700 for mapping an explosive event, according to an example embodiment. In an example, the method 700 can include operations such as: laying out sensor grids (dense meshwork with embedded sensor devices) within an area of interest at 705, optionally time synchronizing the sensor devices within the sensor grids at 710, optionally mapping location of each sensor within the sensor grid(s) at 715, triggering an event within the area of interest at 720, collecting data from all surviving sensors in the sensor grid(s) at 725, analyzing data collected from the sensors at 730, optionally correlating data between neighboring sensors at 735, optionally generating visualizations from the sensor data at 740, and outputting analysis of sensor data and any generated visualizations at 745.

In this example, the analysis of data collected from the sensors at 730 can include a variety of statistical processing steps to analyze the potentially large number of sensors in the grids. At 735, the method 700 can include mathematical algorithms to correlate data between neighboring sensors. Data correlation or statistical analysis between sensors can be used to improve the overall quality of the measurement system by smoothing outliers or averaging results to increase accuracy. An outlier can include a data point that deviates from neighboring data beyond a pre-defined threshold. Data correlation can also be used to fill in data for dead or destroyed sensors in the grid. For example, a high density grid of low resolution sensors can be used to generate regional averages providing a courser spatial density, but higher resolution mapping of an explosive event. A benefit of this approach, is obtaining high resolution data from lower cost sensor devices. Interpolation can be used to fill in measurement gaps for damaged sensors in the high density grid. Time synchronized data collection permits data visualizations and analysis as described above.

Though arranged serially in the example of FIG. 7, other examples may reorder the operations, omit one or more operations, and/or execute two or more operations in parallel using multiple processors or a single processor organized as two or more virtual machines or sub-processors. Moreover, still other examples can implement at least some of the operations as one or more specific interconnected hardware or integrated circuit modules with related control and data signals communicated between and through the modules. Thus, any process flow is applicable to software, firmware, hardware, and hybrid implementations.

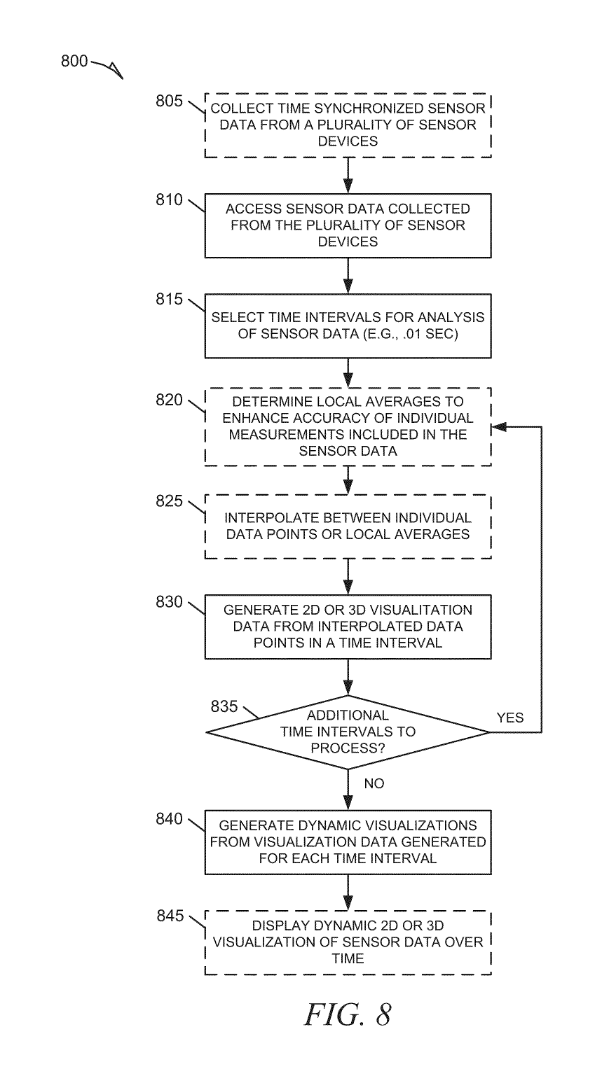

FIG. 8 is a flowchart illustrating a method 800 for dynamically mapping an explosive event using data from a plurality of sensor devices, according to an example embodiment. In this example, the method 800 can include operations such as: optionally collecting sensor data at 805, accessing sensor data at 810, selecting analysis time intervals at 815, optionally determining local averages at 820, interpolating between individual data points at 825, generating visualization data at 830, determining if additional time intervals need to be processed at 835, generating a dynamic visualization at 840, and optionally displaying the dynamic visualization at 845. In an example, the method 800 can being with a data analysis system collecting time synchronized data from a plurality of sensor devices. As discussed above, data collection can be performed via wired or wireless communication interfaces with each individual sensor device. In certain examples, sensor devices in a dense meshwork grid can be wired together, with the meshwork grid wiring providing a communication path to each sensor device in the grid.

At 810, the method 800 can continue with the data analysis system accessing sensor data collected from the plurality of sensor devices. In an example, the sensor data can include data steams at a sampling rate for a plurality of environmental parameters measured during the explosive blast, location data for each sensor device, and timestamp data allowing for synchronization of the data streams.

At 815, the method 800 can continue with the data analysis system receiving a selected time interval for analysis of the collected data. The dynamic mapping of blast data can include a temporal component enabling visualization of how a blast propagated through a test environment. The time interval selection controls the level of temporal detail the dynamic mapping results. For example, if 0.1 seconds is selected, the data analysis system will generate a visualization snapshot everyone 0.1 seconds over the duration of the data collection time frame. Selection of 0.01 seconds as the time interval will produce 100 times more visualization snapshots for the same time frame. The time interval selected is generally not shorter than the period of the sampling frequency of the sensor devices. However, in certain examples, a time interval can be set shorter than the period of the sampling frequency of the sensor devices. In these examples, the data analysis system can perform time-based interpolation to fill-in for the lower frequency data.

At 820, the method 800 can optionally continue with the data analysis system determining local averages to enhance accuracy of individual measurements. In an example using a dense meshwork of low-fidelity sensor devices, various averaging techniques can be employed to lower the data density, while increasing the accuracy. This can be particularly advantageous in situations where a certain percentage of sensor devices are likely to be destroyed during the test, as low-cost sensor devices can be used, while still obtaining high accuracy data. The spatial averaging can be performed on all neighboring devices, or the grid can be broken down into segments of sensor devices, among other options. Certain spatial averaging techniques can increase accuracy, while not reducing density of measurements. For example, for each sensor device measures from the eight (8) neighboring devices can all be averaged and used as the measurement for the center sensor device. In this example, all sensor devices, except those along the edge of the meshwork can have an average value calculated. In examples, where local averages are calculated, the output from this operation can be used throughout the remaining analysis operations.

At 825, the method 800 can continue with the data analysis system interpolating between individual data points within the current time interval being processed. The individual data points can represent an individual measurement from an individual sensor device or a spatially averaged data point that may represent a plurality of sensor devices. Output from the interpolation operation can include an interpolated data set, which can be used in subsequent analysis operations. At 830, the method 800 can continue with the data analysis system generating 2D or 3D visualization data from interpolated data points. In certain examples, the data analysis system can use the interpolated data set, the local averages data set, and the original measurement data, or some other combination of these data sets. In an example, the data analysis system generates a temporal snapshot at operation 830 for the time interval currently being processed.

At 835, the method 800 continues with the data analysis system determining whether there are additional time intervals to process within the data collected from the sensor devices. If there are additional time intervals, the method 800 loops back to operation 820 to process the next time interval. If there are no additional time intervals to process, the method 800 can continue at 840 with the data analysis system generating dynamic 2D or 3D visualizations of the blast data, based on the visualizations generated for each time interval (e.g., snapshots). The 2D and 3D visualization can include the processed data mapped onto a virtual model of the test environment.

At 845, the method 800 can conclude with the data analysis system displaying (or transmitting to a display) the 2D or 3D visualization of blast data. In an example, the display of the dynamic visualizations can include a user interface with controls to move forward or backward in time, allowing for temporal visualization of the explosive event.

Though arranged serially in the example of FIG. 8, other examples may reorder the operations, omit one or more operations, and/or execute two or more operations in parallel using multiple processors or a single processor organized as two or more virtual machines or sub-processors. Moreover, still other examples can implement the operations as one or more specific interconnected hardware or integrated circuit modules with related control and data signals communicated between and through the modules. Thus, any process flow is applicable to software, firmware, hardware, and hybrid implementations.

### Modules, Components and Logic

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within an office environment or as a server farm), while in other embodiments the processors may be distributed across a number of locations. In certain examples, at least a portion of the processor-implemented operations can be performed on the sensor devices, such as sensor device 10.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

### Electronic Apparatus and System

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

### Example Machine Architecture and Machine-Readable Medium

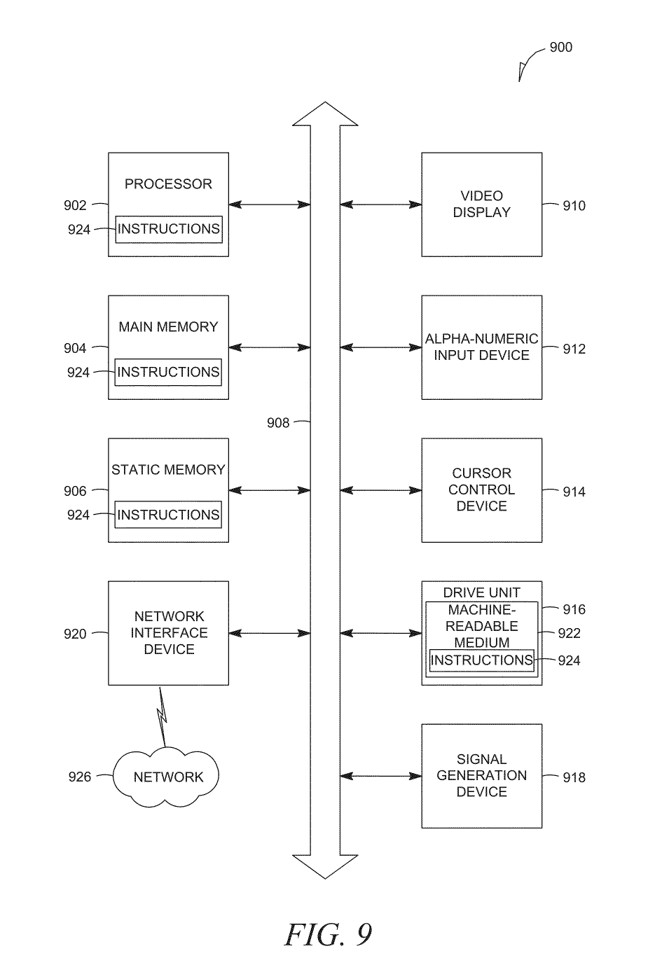

FIG. 9 is a block diagram of machine in the example form of a computer system 900 within which instructions, for causing the machine to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 900 includes a processor 902 (e.g., a central processing unit (CPU), a graphics processing unit (GPU) or both), a main memory 904 and a static memory 906, which communicate with each other via a bus 908. The computer system 900 may further include a video display unit 910 (e.g., a liquid crystal display (LCD) or a cathode ray tube (CRT)). The computer system 900 also includes an alphanumeric input device 912 (e.g., a keyboard), a user interface (UI) navigation device 914 (e.g., a mouse), a disk drive unit 916, a signal generation device 918 (e.g., a speaker) and a network interface device 920.

### Machine-Readable Medium

The disk drive unit 916 includes a machine-readable medium 922 on which is stored one or more sets of instructions and data structures (e.g., software) 924 embodying or used by any one or more of the methodologies or functions described herein. The instructions 924 may also reside, completely or at least partially, within the main memory 904, static memory 906, and/or within the processor 902 during execution thereof by the computer system 900, the main memory 904 and the processor 902 also constituting machine-readable media.

While the machine-readable medium 922 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

### Transmission Medium

The instructions 924 may further be transmitted or received over a communications network 926 using a transmission medium. The instructions 924 may be transmitted using the network interface device 920 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

Although an embodiment has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “second,” and “third,” and so forth are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

The claimed invention includes:

1. A system for dynamic mapping of explosive events within a test environment, the system including:
a plurality of sensor devices, each sensor device including:
a environmental sensor to measure at least one parameter of an explosive event;
a time synchronization circuit to synchronize time stamping of the at least one parameter measured by the environmental sensor; and
a communication interface; and
a data analysis system including a processor and a memory device, the memory device including instructions that, when executed by the data analysis system, cause the data analysis system to:
access measurement data collected from the plurality of sensor devices during an explosive event; and
analyze the measurement data to produce a dynamic visualization of the at least one parameter mapped onto a virtual representation of the test environment.

2. The system of claim 1, wherein the instructions that cause the data analysis system to analyze the measurement data include instructions to divide the measurement data into time intervals based on time stamps included in the measurement data and generate a visualization snapshot for each time interval.

3. The system of claim 2, wherein the instructions that cause the data analysis system to analyze the measurement data include instructions to compile the visualization snapshots for each time interval into the dynamic visualization enabling display of a propagation of the measured parameter throughout the test environment over time.

4. The system of claim 1, wherein the instructions that cause the data analysis system to analyze the measurement data include instructions to interpolate between parameters measured by neighboring sensor devices.

5. The system of claim 1, wherein the plurality of sensor devices are mounted in a meshwork to form a regular grid of sensor devices.

6. The system of claim 5, wherein each sensor device within the regular grid of sensor devices includes its relative position within the grid as part of the measurement data.

7. The system of claim 5, wherein the instructions that cause the data analysis system to analyze the measurement data include instructions to calculate spatial averages for a plurality of locations within the regular grid of sensor devices using parameter measurements from sensor devices within a pre-defined proximity to each location of the plurality of locations.

8. The system of claim 5, wherein the instructions that cause the data analysis system to analyze the measurement data include instructions to calculate spatial averages for at least a sub-set of the plurality of sensor devices within the regular grid of sensor devices using parameter measurements from neighboring sensor devices with corresponding time stamps.

9. The system of claim 1, wherein the instructions that cause the data analysis system to access the measurement data include instructions to access sensor device location data.

10. The system of claim 7, wherein the instructions that cause the data analysis system to analyze the measurement data include instructions to use the sensor device location data to map the measurement data into a virtual map of the test environment.

11. The system of claim 1, wherein each sensor device further includes circuitry and instructions to utilize the wireless communication interface to triangulate a position of the sensor device within the test environment.

12. The system of claim 1, further including multiple wireless base stations for sending and receiving wireless transmissions to the plurality of sensor devices.

13. The system of claim 12, wherein the instructions that cause the data analysis system to access the measurement data include instructions to collect the measurement data via at least one wireless base station from the plurality of sensor devices.

14. The system of claim 12, wherein the memory device further includes instructions that cause the data analysis system to use the wireless base stations to triangulate a position within the test environment for each sensor device of the plurality of sensor devices.

15. A method for dynamic mapping of explosive events within a test environment, the method comprising:
accessing event data of a plurality of data streams, each data stream of the plurality of data streams including a plurality of time stamped environmental measurements from a sensor device within the test environment sampled over a time period and location data for the sensor device within the test environment; and
analyzing, using one or more processors, a plurality of data streams at selected time intervals over the time period to generate a visualization of the explosive event, the analyzing including for each time interval:
generating visualization data representing the time interval based at least in part on portions of the plurality of time stamped environmental measurements representing the time interval, the visualization data mapping the environmental measurements into a virtual representation of the test environment using the location data.

16. The method of claim 15, wherein analyzing the plurality of data streams includes interpolating between time stamp matched environmental measurements from the plurality of data streams, the interpolating occurring between at least two data streams representing neighboring sensor devices.

17. The method of claim 16, wherein interpolating can include interpolating in three dimensions where a neighboring sensor device is spatially distant.

18. The method of claim 15, wherein analyzing the plurality of data streams includes compiling the visualization data generated for each time interval into a dynamic visualization of the explosive event.

19. The method of claim 18, further including displaying the dynamic visualization within a user interface that includes controls to move forward and backward in time through the dynamic visualization.

20. The method of claim 15, wherein analyzing the plurality of data streams includes determining local averages at least in part by calculating an average value from time stamp matched environmental measurements from data streams associated with neighboring sensor devices.

21. The method of claim 18, wherein determining local averages includes reducing data density.

22. The method of claim 15, wherein analyzing the plurality of data streams includes eliminating outliers using statistical analysis.

23. The method of claim 15, wherein accessing the plurality of data streams includes receiving a data stream of the plurality of data streams from each sensor device within the test environment.

24. A machine-readable storage medium including instructions which, when executed by one or more processors, cause the one or more processors to:
access location data associated with a plurality of blast detection devices, the location data identifying a location within a test environment of each blast detection device of the plurality of blast detection devices previously arranged throughout the test environment to capture data from an explosive event within the test environment;
access time stamped environmental measurement data collected from the plurality of blast detection devices, the time stamped environmental measurement data correlated with the location data;
generate, using at least the time stamped measurement data, a visualization depicting a representation of a measured aspect of the explosive event within a virtual representation of the test environment; and
cause the display of the visualization.

25. A system for dynamic mapping of explosive events within a test environment, the system including:
a plurality of sensor devices woven into a wired meshwork interconnecting and positioning the plurality of sensor devices, each sensor device in the wired meshwork including:
a environmental sensor to measure at least one parameter of an explosive event; and
a communication interface;
a data analysis system including a plurality of processor implemented modules, the modules including:
a time synchronization module to synchronize time stamping of event data collected from the plurality of sensor devices;
a data access module to access measurement data collected from the plurality of sensor devices, the measurement data recorded during an explosive test; and
an analysis module to analyze the measurement data to produce a dynamic visualization of the at least one parameter included in the measurement data, the at least one parameter is mapped onto a virtual representation of the test environment.
